(19) United States
(12) Patent Application Publication
(10) Pub. No.: US 2021/0379565 A1
(43) Pub. Date: Dec. 9, 2021

(54) PROCESSES FOR CARRYING OUT CHEMICAL REACTIONS IN FLUID PHASE IN THE PRESENCE OF FILMS COMPRISING CATALYST PARTICLES

(71) Applicant: BASF SE, Ludwigshafen am Rhein (DE)

(72) Inventors: Carlos LIZANDARA PUEYO, Ludwigshafen am Rhein (DE); Lukas WENGELER, Ludwigshafen am Rhein (DE); Sabine HUBER, Ludwigshafen am Rhein (DE); Stephan A. SCHUNK, Heidelberg (DE); Jaroslaw Michael MORMUL, Ludwigshafen am Rhein (DE); Dominique MOULIN, Ludwigshafen am Rhein (DE); Stephan Josef ASCHAUER, Mannheim (DE); Chiara BOSCAGLI, Heidelberg (DE); Marcel SCHMITT, Ludwigshafen am Rhein (DE); Matthias Georg SCHWAB, Ludwigshafen am Rhein (DE); Michael REISER, Ludwigshafen am Rhein (DE)

(21) Appl. No.: 17/282,710

(22) PCT Filed: Sep. 26, 2019
(86) PCT No.: PCT/EP2019/076061

§ 371 Date: Apr. 2, 2021

(30) Foreign Application Priority Data
Oct. 2, 2018 (EP) 18198157.2

(51) Int. Cl. B01J 23/44; B01J 21/18; B01J 31/06; B01J 35/02; B01J 35/06; B01J 8/02; B01J 8/06; C07C 209/36

(52) U.S. Cl. CPC B01J 23/44; B01J 21/18; B01J 31/06; C07C 209/36; B01J 35/06; B01J 8/0278; B01J 8/065; B01J 35/026

(57) ABSTRACT

The present invention relates to a process for carrying out a chemical reaction in a chemical reactor, in which at least one starting material, which is an organic chemical compound comprising 1 to 80 carbon atoms, is converted into at least one reaction product in a fluid phase in the presence of a film comprising solid catalyst particles, which catalyze said chemical reaction, and comprising an organic polymer in fibrillated form, wherein the mass fraction of the sum of the starting material and of the reaction product based on the total mass of the fluid phase is in the range from 0.01 to 1.

The present invention relates to a process for carrying out a chemical reaction in a chemical reactor, in which at least one starting material, which is an organic chemical compound comprising 1 to 80 carbon atoms, is converted into at least one reaction product in a fluid phase in the presence of a film comprising solid catalyst particles, which catalyze said chemical reaction, and comprising an organic polymer in fibrillated form, wherein the mass fraction of the sum of the starting material and of the reaction product based on the total mass of the fluid phase is in the range from 0.01 to 1.

Heterogeneous catalysis plays a central role in the modern chemical industry. Heterogeneous catalysts frequently comprise metals and/or metal oxides with whose surface the reactants in the reaction to be catalyzed interact. Heterogeneous inorganic catalysts usually exist in the form of powders, which provide high surface areas, high catalytic activity, good mass and heat transfer. Since the handling of catalysts in powder form is technically not desired due to separation problems after finishing the chemical reactions, the catalytically active powders are usually converted into bigger shaped bodies e.g. in the form of granules, extrudates, pellets or rings by known methods. The form of the shaped bodies comprising the solid catalyst particles has often to be tailored to the used reactor system in which the chemical reaction takes place.

US 2011/0313186 A1 describes a process for hydrogenation of unsaturated carbon-carbon, carbon-nitrogen or carbon-oxygen bonds, wherein a solid catalyst is used, which is obtained by contacting a monolithic catalyst support with a suspension of a catalytically active transition metal compound. The size of the monolithic catalyst support predetermines the applicable reactor type and the mechanical stability of the used catalytic bodies is not sufficient under reaction conditions, wherein the catalytic bodies are not fixed in the reactor.

U.S. Pat. No. 4,224,185 describes a method for forming shaped, solid catalysts by mixing solid catalyst particles with a fibrillatable polymer, in particular Teflon powder, wherein the fibrillatable polymer is present in an amount from about 0.01 wt. % to about 5 wt. % of the mixture consisting of solid catalyst particles and polymer.

EP 0 057 990 describes a method of making a polymeric catalyst structure comprising a particulate catalyst material encradled in a porous, fibre-containing polymeric material, wherein the final catalyst structure comprises 1 to 5 weight % of a fibrillated polymer, such as PTFE.

U.S. Pat. No. 4,358,396 discloses a particulate catalyst composition adaptable for use in a fixed or fluidized catalyst bed, wherein the particulate catalyst composition is composed essentially of an active (or activatable) material, a fibrillated first polymer and a support-contributing second polymer.

Chen Yijun et al., Journal of Colloid and Interface Science 491 (2017), 37-43 discloses the preparation of Au nanocrystals in the presence of alpha-zein in fibrillated form.

Renliang Huang et al., Environ. Sci. Technol. 2016, 50, 11263-11273 discloses a catalytic membrane reactor, which contains a membrane matrix and a catalytic film of alloy nanoparticle-loaded protein fibrils for continuous reduction of 4-nitrophenol.

Proceeding from the prior art, a first object of the invention is to provide a process for carrying out a chemical reaction in a wide variety of chemical reactor types using a catalyst system showing similar catalytic activity as catalysts in powder form, so called suspension catalysts, without the disadvantages of these catalysts in powder form or without of the limited flexibility associated with the use of bigger shaped catalyst bodies comprising catalytic active components, which additionally often suffer in terms of catalytic performance compared with the corresponding suspension catalyst.

A second object of the invention is to provide improved films comprising solid catalyst particles, which catalyze a chemical reaction, wherein the improved films show better mechanical stability which is necessary to improve the economical overall performance of the chemical processes.

A further object of the invention is to provide new catalyst systems, which show similar catalytic activities as suspension catalysts but do not show the disadvantages of suspension catalysts with respect to work up problems after finishing the chemical reaction.

This object is achieved by a process for carrying out a chemical reaction in a chemical reactor, in which at least one starting material, which is an organic chemical compound comprising 1 to 80 carbon atoms, is converted into at least one reaction product in a fluid phase in the presence of a film comprising solid catalyst particles, which catalyze said chemical reaction, and comprising an organic polymer in fibrillated form, wherein the mass fraction of the sum of the starting material and of the reaction product based on the total mass of the fluid phase is in the range from 0.01 to 1, preferably 0.02 to 1, more preferably 0.04 to 1, in particular 0.1 to 1.

This object is also achieved by a process for carrying out a chemical reaction in a chemical reactor, in which at least one starting material, which is an organic chemical compound comprising 1 to 80 carbon atoms, is converted into at least one reaction product in a fluid phase in the presence of a film comprising solid catalyst particles, which catalyze said chemical reaction, and comprising an organic polymer in fibrillated form, wherein the film comprises at least one layer comprising solid catalyst particles and the organic polymer in fibrillated form, wherein the mass fraction of the organic polymer in fibrillated form in said layer is in the range from 0.06 to 0.2 and the mass fraction of solid catalyst particles in said layer is in the range from 0.8 to 0.94 based on the total weight of said layer, wherein the organic polymer is a fluoropolymer, and wherein the mass fraction of the sum of the starting material and of the reaction product based on the total mass of the fluid phase is in the range from 0.01 to 1, preferably 0.02 to 1, more preferably 0.04 to 1, in particular 0.1 to 1.

The chemical reactions which can be carried out in the inventive process can be varied in a wide range and they are in principle known to the person skilled in the art.

Preferably the chemical reaction carried out in the inventive process is selected from the group of chemical reactions consisting of oxidations, reductions, substitutions, additions, eliminations and rearrangements, more detailed consisting of oxidations, hydroxylations, ammoximations, ammoxidation reactions, epoxidations, aminations, reductions, hydrogenations, dehydrogenations, isomerizations, dehydrations, hydrations, hydrogenolysis reactions, (hydro)halogenations, de(hydro)halogenations, oxyhalogenations, nitrations, denitrifications, (trans)alkylations, dealkylations, disproportionation reactions, acylations, alkoxylations, substitutions, additions, eliminations, esterifications, transesterifications, hydrocyanations, hydroformylations, carbonylations, methylations, condensations, aldol condensations, metathesis, dimerizations, oligomerizations, polymerizations, rearrangements and enzymatic reactions, preferably selected from the group of chemical reactions consisting of hydrogenations.

A general system of nomenclature for transformations whereby one organic compound is converted into another compound is described in Pure & Appl. Chem., Vol. 61, No. 4, pp. 725-768, 1989 in the articles “NOMENCLATURE FOR ORGANIC CHEMICAL TRANSFORMATIONS”. In this systematic approach, the term transformation is used instead of the term “chemical reaction”, which is used in the present description of the invention and which comprises “transformations” as well as “reactions” in the sense of the cited article.

In one embodiment of the present invention the inventive process is characterized in that the chemical reaction is selected from the group of chemical reactions consisting of oxidations, reductions, substitutions, additions, eliminations and rearrangements.

In one embodiment of the present invention the inventive process is characterized in that the chemical reaction is selected from the group of chemical reactions consisting of hydrogenations.

The temperature of the chemical reaction, which is carried out in the inventive process, can be varied in a wide range. Preferably the chemical reaction takes place at a temperature in the range from −78° C. to 350° C., more preferably in the range from −10° C. to 300° C., more preferably in the range from 10° C. to 200° C.

In one embodiment of the present invention the inventive process is characterized in that the chemical reaction takes place at a temperature in the range from −78° C. to 350° C.

The chemical reactors in which the inventive process can be carried out can be varied in a wide range. The different chemical reactors for carrying out above-mentioned different chemical reactions are known to the person skilled in the art. Examples of reactors, which are suitable for heterogenous catalytic reactions are fixed bed reactors, moving bed reactors, rotation bed reactors, fluidized bed reactors or slurry reactors. Preferably the chemical reactor is a fixed-bed reactor selected from the group of reactors consisting of tubular reactors, adiabatic reactors, multitube reactors and microreactors.

In one embodiment the reaction in the fixed bed reactor is carried out in trickle bed mode.

In one embodiment the reaction in the fixed bed reactor is carried out in sump mode in upstream or downstream variant with concurrent flow of gas and liquid.

In one embodiment the reaction in the fixed bed reactor is carried out in sump mode in upstream or downstream variant with countercurrent flow of gas and liquid.

In one embodiment the catalyst is placed inside a stirred tank vessel by a suitable arrangement of fixation devices.

In one embodiment of the present invention the inventive process is characterized in that the chemical reactor is a fixed-bed reactor selected from the group of reactors consisting of tubular reactors, adiabatic reactors, multitube reactors and microreactors.

The above-mentioned chemical reactors can be operated batch-wise or in continuous flow conditions. Preferably the chemical reactor is operated under continuous flow conditions.

In one embodiment of the present invention the inventive process is characterized in that the chemical reactor is operated under continuous flow conditions.

The person skilled in the art can usually determine directly the nature of the reaction product or the main reaction product and potential side products based on the used starting material, the reaction type and the applied reaction conditions including the used catalyst system.

In the inventive process the conversion of the starting material to a reaction product takes place in a fluid phase, either in a gaseous phase or in a liquid phase, preferably in a liquid phase. Preferably, the starting material, i.e. the organic compound, is itself a liquid or a gas under the reaction conditions or it is soluble in an inert medium, which itself is a liquid or a gas, preferably liquid under the reaction conditions.

In one embodiment of the present invention the inventive process is characterized in that the fluid phase is a liquid phase.

In case the starting material is reacted in a liquid phase with a reagent, which is a gas under “Standard Temperature and Pressure” conditions (298.15 K; 1,000 bar), such as hydrogen or oxygen, these gaseous reagents form preferably a third phase in the reactor, beside the solid phase of the film and the liquid phase comprising the starting material and they dissolve partially in the liquid phase or they are absorbed by the solid catalyst particles of the film.

In one embodiment of the present invention the inventive process is characterized in that the chemical reactor comprises a fluid phase, which comprises the starting material, and a gaseous phase, which comprises a reagent, which is a gas under “Standard Temperature and Pressure” conditions is a liquid phase.

In one embodiment the reaction is carried out as two phase reaction gas-solid.

In one embodiment the reaction is carried out as two phase reaction liquid-solid.

In one embodiment the reaction is carried out as three phase reaction gas-liquid-solid.

In another embodiment the reaction is carried out under supercritical conditions with the catalyst being present in solid phase.

The chemical reaction, which is carried out in the inventive process, is performed in the presence of a film comprising solid catalyst particles, which catalyze said chemical reaction, and comprising an organic polymer in fibrillated form.

The film comprises, as a first component, solid catalyst particles of at least one catalytically active material, which catalyze above-described chemical reactions.

In the context with the present invention the term “solid catalyst particles” includes beside particles comprising any material which exhibits catalytic activity without needing any activation step also particles comprising pre-cursors of a catalytically active material, which must be activated at the latest in the chemical reaction itself to become a catalytically active material. For example, PtO₂ is first reduced by hydrogen to Pt, before the hydrogenation of an olefin takes place catalyzed by Pt metal.

Solid catalyst particles, which catalyze above-described chemical reactions are known to the person skilled in the art. The solid catalyst particles may consist essentially of a catalytically active material itself or its to be activated precursor or the solid catalyst particles comprise a support material, which is usually catalytically inactive, and which is preferably porous, and the catalytically active material or its to be activated precursor, which is deposited on the surface of said support material, preferably on the inner and outer surface of said porous support material (e. g. palladium on activated carbon).

In one embodiment, the catalytically active material comprises a metal or metal compound.

Preferably, the catalytically active material comprises one or more metals selected from the group consisting of Li, Na, K, Rb, Cs, Mg, Ca, Sr, Ba, Al, Ga, Si, Sn, Pb, P, Sb, Bi, Ti, Zr, Hf, V, Nb, Ta, Cr, Mo, W, Mn, Re, Fe, Ru, Os, Co, Rh, Ir, Ni, Pd, Pt, Cu, Ag, Au, Zn, Cd, Hg, lanthanide metals or actinide metals, or metal compounds, preferably selected from the group of metal compounds consisting of oxides, peroxides, superoxides, hyperoxides, nitrides, carbides, sulfides, nitrates, (poly)phosphates, sulfates, (poly)tungstates, (poly)molbydates, aluminates, alumino-silicates, titanates, halogenides, hydroxides, carbonates, hydroxycarbonates, or mixtures thereof.

Metal oxides may comprise a single or mixed metal oxide such as a spinel or perovskite, or a composition comprising two or more metal oxides.

In one embodiment the catalytically active material comprises a precious metal, e. g. one or more of Pt, Pd, Ir, Ru, Os, Re, Rh, Au, Ag, optionally mixed with one or more additional metals and/or metal compounds, and optionally deposited on the surface of a porous support material.

In one embodiment the catalytically active material comprises a material which is pyrophoric (e. g. Raney-Nickel).

In one embodiment the catalytically active material comprises a zeolite.

In one embodiment the catalytically active material comprises a clay.

In one embodiment the catalytically active material comprises an organic compound such as an organic polymer. Suitable organic compounds are acidic, alkaline or ion-exchange resins such as polystyrene sulfonate resins (e. g. Amberlyst) or sulfonated tetrafluoroethylene based fluoropolymer-copolymers (e. g. Nafion).

In one embodiment the catalytically active material comprises an enzyme.

The catalytically active material may further comprise one or more support materials such as alumina, silica, titania, zirconia, silicon nitride, silicon carbide, activated carbon, carbon black, graphite, carbon nanotubes, graphene, cordierite, ceramics and mixtures thereof.

Suitable combinations are e. g. a precious metal supported on alumina, silica, activated carbon or carbon black.

The solid catalyst particles, which are used in the inventive process, are preferably porous, wherein the porosity can be varied in a wide range. Preferably the porosity of the solid catalyst particles is characterized by a specific surface area determined by the BET method, wherein the specific surface area is in the range of 1 to 3000 m²/g, preferably in the range of 2 to 1000 m²/g, most preferably in the range of 10 to 500 m²/g.

In one embodiment of the present invention the inventive process is characterized in that the solid catalyst particles have a specific surface area determined by the BET method, which is in the range of 1 to 3000 m²/g, preferably in the range of 2 to 1500 m²/g, most preferably in the range of 10 to 500 m²/g.

In one embodiment of the present invention the inventive process is characterized in that the solid catalyst particles have a particle size d50 in the range from 0.1 to 1000 μm, preferably from 1 to 500 μm, from 5 to 300 μm, more preferably from 10 to 250 μm. The particle size d50 is determined by the laser diffraction method according to ISO 13320 on a Mastersizer 2000 (Malvern Instruments Ltd.).

The film comprises as a second component an organic polymer in fibrillated form. Organic polymers, which are capable of being fibrillated (“fibrillatable”), preferably during a process step for preparing the film for the inventive process, are known to the person skilled in the art. “Fibrillation” is a term of art that refers to the treatment of suitable polymers to produce, for example, a “node and fibril” network, or cage-like structures.

The mass fraction of the organic polymer in fibrillated form applied in the film as a second component can be varied in a wide range. Preferably, the mass fraction is in the range from 0.06 to 0.2, more preferably in the range from 0.07 to 0.15, in particular the range from 0.08 to 0.12 based on the total weight of the film.

In one embodiment of the present invention the inventive process is characterized that the mass fraction of the organic polymer in fibrillated form applied in the film as a second component is in the range from 0.06 to 0.2, more preferably in the range from 0.07 to 0.15, in particular the range from 0.08 to 0.12 based on the total weight of the film.

Suitable organic polymers, which can be fibrillated are known to the person skilled in the art. Preferably the organic polymer is selected from the group consisting of fluoropolymers, ultrahigh-molecular weight polyethylene and polyethylene oxides, preferably fluoropolymers, in particular polytetrafluoroethylene.

Fibrillated fluoropolymers are known in the art. Starting from a fluoropolymer capable of processing-induced fibrillation the polymer, optionally as part of a mixture, is subject to sufficient shear force to induce fibrillation.

It is clear to the practitioner in the art that film can comprise one, two different or more different polymers, in particular different fluoropolymers. Accordingly, the term “an organic polymer in fibrillated form” refers to one or more fibrillatable polymers, for example two, three or four, fluoropolymers. Any given weight percentages given for the organic polymer in fibrillated form refer to all fibrillatable polymers, preferably fluoropolymers in the layer and thus can be calculated from the sum of said polymers. However, it is preferred that only one fibrillatable polymer, preferably one fluoropolymer is present in the film.

In a preferred embodiment the at least one fibrillated fluoropolymer is selected from the group of polymers and copolymers consisting of trifluoroethylene, hexafluoropropylene, monochlorotrifluoroethylene, dichlorodifluoroethylene, tetrafluoroethylene, perfluorobutyl ethylene, perfluoro-(alkyl vinyl ether), vinylidene fluoride, and vinyl fluoride and blends thereof. Accordingly, homopolymers of one of the above monomers or copolymers of two or more of these monomers can be used. More preferably, the organic polymer in fibrillated form is a fibrillated polytetrafluoroethylene (PTFE).

In one embodiment of the present invention the inventive process is characterized in that the organic polymer is a fluoropolymer, in particular polytetrafluoroethylene.

Such fibrillated PTFE is known in the art, e.g. from US 2003/0219587 A1, U.S. Pat. No. 4,379,772 A, US 2006/0185336 A1, U.S. Pat. Nos. 4,153,661 A and 4,990,544. Accordingly, it has been recognized that, when subjected to shear forces, small particles of certain polymeric materials, e. g., per-fluorinated polymers such as PTFE, will form fibrils of microscopic size. Using this knowledge, Ree et al. described in the late 1970s in U.S. Pat. No. 4,153,661 A a PTFE composite sheet for use as an electronic insulator, a battery separator, and/or a semipermeable membrane for use in separation science. Formation of the tough, attractive, and extremely pliable film involved intensive mixing of the PTFE and lubricant mixture sufficient to cause the PTFE fibrils to fibrillate and form a sheet. Thus, the fibrils of polytetrafluoroethylene resin can be obtained by applying a shearing stress to particles of polytetrafluoroethylene resin (US 2006/0185336 A1). U.S. Pat. No. 4,990,544 relates to a composition comprising fibrillated polytetrafluoroethylene resin and a fine inorganic powder. Said composition is used as a gasket material.

The PTFE resin can have a number average molecular weight of 3,000,000 to 50,000,000 g/mol, preferably from 5,000,000 to 15,000,000 g/mol, as described in US 2006/0185336 A1

Suitable PTFE grades are commercially available such as Teflon manufactured by The Chemours Company; Fluon manufactured by ASAHI GLASS CO., LTD. of Japan and Dyneon manufactured by the 3M Company, St. Paul, Minn.

The film can be freestanding, folded to itself or supported. In case the film is supported any suitable support can be used. Such support can be porous, partly porous or non-porous. The support can be mono- or multi-layered. The support can be thermally and/or electrically conducting, semiconducting or insulating. A rigid or flexible support is possible. Examples for suitable supports include metals, like titanium, zirconium, hafnium, vanadium, niobium, tantalum, chromium, molybdenum, tungsten, manganese, rhenium, iron, ruthenium, osmium, cobalt, rhodium, iridium, nickel, platinum, palladium, copper, silver, gold, zinc, aluminum, tin, lead, metals from the lanthanide series; metal alloys like steel; carbon substrates; meshes; fabrics; cellulose materials, like paper and wood; ceramics; semi-conductors, like silicon, germanium, gallium arsenide, indium phosphide, glass; quartz; metal oxides, like aluminum oxide, silicon oxide, zirconium oxide and indium tin oxide; silicon carbide; polymers and the like.

In one embodiment of the present invention the inventive process is characterized in that the film is freestanding.

In another embodiment of the present invention the inventive process is characterized in that the film is supported.

The film can be supplied to the support after its preparation by suitable deposition methods. Examples for such methods adhesion coating using an adhesive, which can also be part of the film as an additive component (C) or only using adhesion forces of the film when stamping, pressing, molding or embossing the film onto the support.

In one embodiment the support can be supplied in the form of a net-like or mesh structure, supporting the film from one or two sides.

In one embodiment two nets of the support hold the film by sufficient mechanical force.

In one embodiment the film and the supporting net-like structure can be supplied as rolled composition in which the net-like structure acts as spacer between the rolled film.

The thickness of the film used in the inventive process can be varied in a wide range. Preferably, the thickness of the film is in the range from 0.1 μm to 20000 μm, more preferably in the range from 1 μm to 7500 μm, in particular the range from 10 μm to 5000 μm.

In one embodiment of the present invention the inventive process is characterized in that the film has a thickness in the range from 0.1 μm to 20000 μm.

When the film is freestanding, the film has a thickness of at least 0.5 μm, more preferably 1 μm to 2 cm, even more preferably 1 μm to 1 cm, even more preferably 5 μm to 500 μm, even more preferably 10 μm to 100 μm. When the film is supported, the film has a thickness of at least 0.1 μm, more preferably 1 μm to 2 cm, even more preferably 1 μm to 1 cm, even more preferably 5 μm to 500 μm, even more preferably 10 μm to 100 μm.

In one embodiment of the present invention the inventive process is characterized in that the film has a thickness of at least 0.5 μm when film is freestanding and at least 0.1 μm when the film is supported.

In case the film has different thicknesses the lower value of a range represents the minimum value of all thickness values and the upper value of a range represents the maximum value of all thicknesses.

Preferably, the film has a two-dimensional surface with at least one dimension which exceeds 1 cm. The length of the film can be adjusted as required for a specific application. In principle the length is not limited. Thus, also a supply in coils of films is possible. In this case it is advantageous to separate each film layer from each other by separation means, like a release agent or separating foil.

In one embodiment of the present invention the inventive process is characterized in that the film has a two-dimensional surface with at least one dimension with exceeds 1 cm.

Preferably, the film of the present invention comprises pores, in particular micropores and/or mesopores. Micropores are defined as pores having a diameter of 2 nm or less and mesopores are defined by a diameter in the range from 2 to 50 nm (Pure 8 Appl. Chem. 57 (1985) 603-619). The presence of micropores and/or mesopores can be checked by means of sorption measurements, with these measurements determining the uptake capacity of the film for nitrogen at 77 Kelvin in accordance with DIN 66134:1998-02.

Preferably, the specific surface area of the film measured according to BET (DIN ISO 9277:2003-05) is in the range of 1 to 3000 m²/g, preferably 2 to 1500 m²/g, most preferably 10 to 500 m²/g.

Preferably, the volumetric specific surface area of the film is between 1 and 15,000 m²/cm³, preferably between 2 and 7000 m²/cm³, most preferably between 10 and 2500 m²/cm³. The volumetric specific surface area can be calculated by determining the product of specific surface area [m²/g] of the film and the density [g/cm³] of the film.

Preferably, the film is preferably flexible. Accordingly, the film can be bended, twisted, rolled, folded or presented as flat film.

In one embodiment of the present invention the inventive process is characterized in that the film is flexible.

In one embodiment of the present invention the inventive process is characterized in that the film is corrugated.

In one embodiment of the present invention the inventive process is characterized in that the film is embossed.

In one embodiment of the present invention the inventive process is characterized in that the film is built up from several layers of alternately planar and corrugated layers forming parallel channels.

The film can be arranged in the chemical reactor in a wide variety of arrays, due to the mechanical characteristics of the film. For example, rolls of a single film or stacks of multiple films can be formed, wherein preferably the surfaces of the film are maintained accessible by introducing appropriate spacer means in the respective arrangement.

In one embodiment of the present invention the inventive process is characterized in that multiple films are arranged to a stack, preferably to a stack of a thickness up to 10 cm, preferable up to 5 cm, more preferably up to 2 cm, in particular up to 1 cm.

The film, as described above, which comprises solid catalyst particles, which catalyze said chemical reaction, and comprises an organic polymer in fibrillated form, film can be built up by one single layer or by several layers, which are preferably not identical in view of all their properties; in particular in view of their composition. For example, one layer comprises solid catalyst particles, while a second layer comprises catalytically inactive solid particles or no solid inorganic particles at all.

The composition of the catalytically active layer of the film, which comprises solid catalyst particles and the organic polymer in fibrillated form, can be varied in a wide range. Preferably the mass fraction of the organic polymer in fibrillated form in said catalytically active layer is in the range from 0.001 to 0.2, preferably in the range from 0.06 to 0.2, more preferably in the range 0.7 to 0.15, in particular in the range from 0.08 to 0.12 and the mass fraction of solid catalyst particles in said catalytically active layer is in the range from 0.8 to 0.999, preferably in the range from 0.8 to 0.94, more preferably in the range 0.85 to 0.93, in particular in the range from 0.88 to 0.92 based on the total weight of said layer.

In one embodiment of the present invention the inventive process is characterized in that the film comprises at least one layer comprising solid catalyst particles and the organic polymer in fibrillated form, wherein the mass fraction of the organic polymer in fibrillated form in said layer is in the range from 0.001 to 0.2, preferably in the range from 0.06 to 0.2, more preferably in the range 0.7 to 0.15, in particular in the range from 0.08 to 0.12 and the mass fraction of solid catalyst particles in said layer is in the range from 0.8 to 0.999, preferably in the range from 0.8 to 0.94, more preferably in the range 0.85 to 0.93, in particular in the range from 0.88 to 0.92 based on the total weight of said layer.

The thickness of the layer, which comprises solid catalyst particles and the organic polymer in fibrillated form can be varied in a wide range. Preferably, the thickness of the layer is in the range from 0.1 μm to 1000 μm, more preferably in the range from 1 μm to 500 μm, in particular in the range from 5 μm to 200 μm.

In one embodiment of the present invention the inventive process is characterized in that the thickness of the layer comprising solid catalyst particles and the organic polymer in fibrillated form is in the range from 1 μm to 200 μm.

In one embodiment of the present invention the inventive process is characterized in that the film comprises at least two layers of different compositions, wherein at least one of the two outer layers of the film is the layer comprising solid catalyst particles and the organic polymer in fibrillated form.

The porosity of the film used in the inventive process can be varied in a wide range. Methods for adjusting the porosity of a film during the formation of a film are known to the person skilled in the art. Usually a pre-film is formed, which comprises an easily removable component, such as a water-soluble salt (e.g. sodium chloride) or a water-soluble polymer (e.g. a solid polyethylenglycol). The final film with an adjusted porosity is obtained after removing the removable component. Preferably, at least of part of the film used in the inventive process provides a porosity of 5 to 70%, preferably 10 to 50%, more preferably 20 to 40%.

In one embodiment of the present invention the inventive process is characterized in that at least one part of the film provides a porosity of 5 to 70%.

The porosity is determined by nitrogen physisorption, mercury pore volume and helium density. It can be determined by the following formula. Porosity (%)=100−[(density of film/film material)×100]. The density of the film is determined by dividing its total weight by its total volume. The density of the film material is determined by measuring mercury pore volume and helium density.

The films, which comprise an organic polymer in fibrillated form and solid catalyst particles, which catalyze said chemical reaction carried out in the inventive process, can be prepared by methods known to the person skilled in the art.

Preferably, the film is prepared by a process comprising the process steps of
a) preparing a mixture comprising solid catalyst particles and at least one fibrillatable organic polymer,
b) fibrillating the organic polymer by applying shear forces and pressure to the mixture prepared in process step a),
c) converting the mixture obtained in process step b) to a film by a film formation step, and
d) optionally further conditioning the primary formed film obtained in process step c).

The description and preferred embodiments of the film and its components, in particular the description of solid catalyst particles as a first component and of the organic polymer, which is fibrillatable, as a second component, in the film forming process correspond to the above description of these components for the film used in the inventive process for carrying out a chemical reaction as described above.

In process step a) a mixture comprising solid catalyst particles and a fibrillatable organic polymer is prepared. Depending on the intended structure and properties of the film, e.g. adjusting a desired porosity, further components, such as the above describe removable pore forming soluble or volatile additives, which are soluble or volatile, like sodium chloride, might be added. The components of the mixture are preferably in pulverous form to achieve easily a homogeneous mixture of the compounds without the need to pulverize any of them.

The preparation of the mixture comprising solid catalyst particles and a fibrillatable organic polymer can be done in the present or absence of a solvent, preferably a solvent with a boiling point below 110° C. Preferably the preparation of the mixture is done without the addition of any solvent to obtain a dry mixture of the components.

In one embodiment of the present invention the inventive process is characterized in that the film is prepared by a process comprising process steps, wherein a dry mixture comprising solid catalyst particles and a fibrillatable organic polymer is converted into a film comprising solid catalyst particles and the organic polymer in fibrillated form.

The organic polymer used in process step a) is a fibrillatable organic polymer, which is not fibrillated, already at least partly fibrillated or fully fibrillated, preferably not fibrillated or already at least partly fibrillated, in particular not fibrillated, before contacted with the solid catalyst particles.

In process step b) the organic polymer is fibrillated by applying shear forces and pressure to the mixture prepared in process step a).

The fibrillation of the organic polymer in process step b) can be achieved by applying pressure and shear, preferably applying pressure and shear at the same time.

Apparatuses, which can be used for the fibrillation step are known to the person skilled in the art. Examples of such apparatuses are mills, preferably a ball mills, mixers or kneaders.

In principle, process step a) and process step b) can be executed one after another or in parallel, that means at the same time.

A suitable mixer is any mixer or kneader that can subject the mixture to sufficient shear forces to fibrillate the fibrillatable organic polymer at the desired processing temperature. Exemplary commercially available batch mixers include the Banbury mixer, the Mogul mixer, the C. W. Brabender Prep mixer, and C. W. Brabender sigma-blade mixer. Known mixer types are Ribbon Blender, V Blender, Continuous Processor, Cone Screw Blender, Screw Blender, Double Cone Blender, Double Planetary, High Viscosity Mixer, Counter-rotating, Double & Triple Shaft, Vacuum Mixer, High Shear Rotor Stator, Dispersion Mixers, Paddle, Jet Mixer, Mobile Mixers, Drum Blenders, Banbury mixer, intermix mixer, Planetary mixer.

In process step c) the mixture obtained in process step b) is converted to a film by a film formation step.

Methods for preparing films starting from dry mixture of solid materials, in particular from mixtures comprising thermoplastic organic polymers are known to the person skilled in the art.

In process step c) preference Is given to film formation processes, wherein the level of the fibrillation of the organic polymer either increased or is maintained but does not decrease. Preferred film formation processes are a calendering process, or any other roll process with at least one roll that applies shear forces and compresses the mixture.

For increasing the fibrillation of the organic polymer, the film might be processed by more than one calendering steps and at least on folding step before the last calendering step.

In one embodiment of the present invention the inventive process is characterized in that the film is folded at least one time.

In principle, process step b) and process step c) can be executed one after another or at the same time, if during the film formation step the fibrillatable polymer can be sufficiently fibrillated and during process step a) the fibrillation grade of the fibrillatable polymer was not significantly increased by avoiding pressure and shear during the preparation of the mixture. Preferred is a process, wherein process step b) and process step c) are executed one after another.

In one embodiment of the present invention the inventive process is characterized in that the film is prepared by the above described process, wherein process steps a) b) and c) are performed in the absence of any solvent.

In optional process step d) the primary formed film obtained in process step c) is further conditioned. Further conditioning can be any further mechanical processing step such as a compressing step or embossing step or stretching step or a thermal treatment step, such as a heating or cooling step. Further conditioning can also be a lamination step of several primary formed films to a composite structure by e.g. calendering rollers or any other lamination process. The composite structure can consist of films which differ in their composition and properties, but can also consist of similar films. Further conditioning can also be a washing step to remove optional, soluble or volatile additives which were added in process step a) for adjusting a desired porosity. The washing of the film can be realized by the insertion into a liquid or a thermal treatment to remove volatile additives or both.

More particularly, the inventive process for carrying out a chemical reaction as described above is suitable for industrial production of desired reaction products, wherein the production volumes are more than 100 kg/day, better more than 1000 kg/day, even better more than 10 t/day or more than 100 t/day.

In one embodiment of the present invention the inventive process is characterized in that the reaction product is produced in a production volume of more than 100 kg/day, better more than 1000 kg/day, even better more than 10 t/day or more than 100 t/day.

The inventive process for carrying out a chemical reaction as described above is also particularly suitable for high throughput experimentation to determine for a certain reaction the most suitable catalyst and optimized reaction conditions.

In one embodiment of the present invention the inventive process is characterized in that the process is performed in a high throughput experimentation system with microreactors.

A further object of the invention is to provide new catalyst systems, which show similar catalytic activities as suspension catalysts but do not show the disadvantages of suspension catalysts with respect to work up problems after finishing the chemical reaction.

The present invention further provides a catalyst system, comprising a film which comprises solid catalyst particles, which catalyze a desired chemical reaction, and an organic polymer in fibrillated form.

The description and preferred embodiments of the film and its components, in particular the solid catalyst particles and the organic polymer in fibrillated form, correspond to the above description of the film, its structure and its components used in the inventive process for carrying out a chemical reaction.

In one embodiment of the present invention the inventive catalyst system is characterized in that the solid catalyst particles catalyze hydrogenation reactions with molecular hydrogen, preferably selected from solid particles comprising Ni, Pd, Pt, Rh, Ru, Co, Cu—Cr and Zn—Cr, more preferably selected from the group of hydrogenation catalysts consisting of Raney nickel, Raney cobalt, Ni on silica, Pd on carbon (Pd/C), Pd-oxide, Pd on CaCO₃, Pd on BaSO₄, Pd on alumina, Pt on carbon (Pt/C), PtO₂ and platinum black, in particular Pd on carbon.

In one embodiment of the present invention the inventive catalyst system is characterized in that the film is build up by one single layer, which has a porosity in the range from 20 to 40%.

In one embodiment of the present invention the inventive catalyst system is characterized in that the film is build up by three layers, wherein the two outer layers comprise solid catalyst particles and the thickness of both outer layers is in the range from 0.1 μm to 200 μm, preferably in the range from 1 μm to 100 μm, in particular in the range from 5 μm to 50 μm.

The above described catalyst systems show similar catalytic activities and performance as the corresponding suspension catalysts but do not show the disadvantages of suspension catalysts with respect to work up problems after finishing the chemical reaction. The films show the desired mechanical stability (macroscopically and microscopically) as confirmed after its use in the inventive process by SEM.

The invention is illustrated by the examples which follow, but these do not restrict the invention.

Figures in percent are each based on % by weight, unless explicitly stated otherwise.

### I. Preparation and Characterization of Films Comprising Solid Catalyst Particles and PTFE in Fibrillated Form

Three catalyst powders (5% Pd on activated carbon) were used as solid catalyst particles. All catalysts were supplied by BASF for hydrogenation of nitrobenzene to aniline. The three powders are here indicated as S1, S2, S3 and they are commercially produced. The three catalysts differ in terms of catalytic activity.

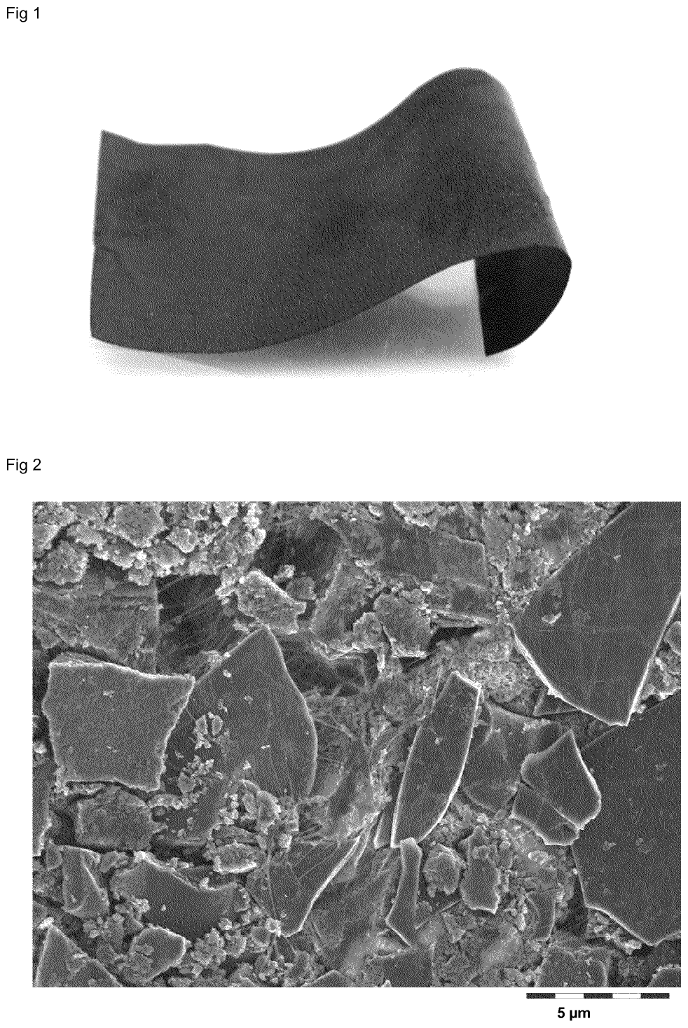

Films comprising solid catalyst particles and PTFE in fibrillated form were shaped by mixing the catalyst powder with a low amount of PTFE (7.5% PTFE) as fibrillatable organic polymer and processed by a sequence of mechanical treatments (kneading, calendering and conditioning). The resulting films have flexible and porous structure (FIG. 1). For each catalyst, three film thicknesses were prepared: 100, 250 and 400 μm (summary in Table 1). For S3 sandwich films were also produced. With the term sandwich films, it is indicated a film constituted of external layers containing the active metal (Pd) on a support material and an internal layer containing only the support without active metal (active carbon).

TABLE 1
Overview of the films prepared and tested.

| | | Thickness of the film |
|---|---|---|
| | | comprising Solid Cat-alyst |
| Sample | | Particles and PTFE in |
| name | Solid Catalyst Particles | fibrillated form |
| S1 | 5% Pd on C (high active) | — |
| S2 | 5% Pd on C (middle active) | — |
| S3 | 5% Pd on C (low active) | — |
| F1 | 5% Pd on C (high active) | 100 μm |
| F2 | 5% Pd on C (middle active ) | 100 μm |
| F3 | 5% Pd on C (low active ) | 100 μm |
| F4 | 5% Pd on C (high active ) | 250 μm |
| F5 | 5% Pd on C (middle active) | 250 μm |
| F6 | 5% Pd on C (low active ) | 250 μm |
| F7 | 5% Pd on C (high active ) | 400 μm |
| F8 | 5% Pd on C (middle active ) | 400 μm |
| F9 | 5% Pd on C (low active) | 400 μm |
| F10 | 5% Pd on C (low active) | Sandwich Film 25 μm |
| | | (active layer)/50 |
| | | μm (inert)/25 μm |
| | | (active layer) |
| F11 | 5% Pd on C (low active) | Sandwich Film 25 μm |
| | | (active layer)/60 |
| | | μm (inert)/25 μm |
| | | (active layer) |

F10 (Table 1) was produced compressing a 315 μm active-carbon layer between 150 μm Pd/C film in order to obtain a final thickness of 100 μm. In FIG. 4 the profile of a sandwich film measured by SEM produced by higher volume compression is reported.

FIG. 1: Image of a film prepared according to example I.

FIG. 2: SEM image of F1. Overview of the film comprising PTFE fibrils and catalyst particles.

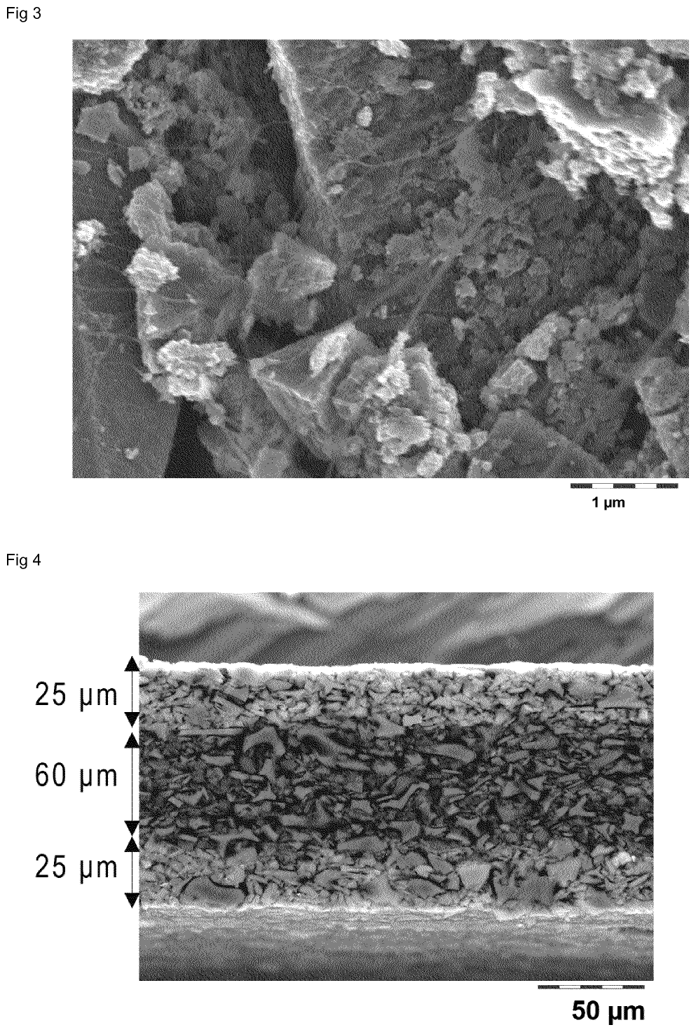

FIG. 3: SEM image of F1. Detail of PTFE nanofibrils keeping together the catalyst particles.

FIG. 4: SEM images of the vertical profile of the sandwich film F11. For this image the film was cut by FIB (Focused Ion Beam) and measure in backscattering mode. The bright regions contain Pd.

The physical-chemical characteristics of the used catalyst powders and the films derived from the highest active catalyst S1 are compared in Table 2.

TABLE 2
Physical and chemical characteristics of the three BASF catalyst
samples S1, S2 and S3 and of the films produced from solid
catalyst particles S1 (catalyst with the highest activity).

| | S1 | S2 | S3 | F1 | F4 | F7 |
|---|---|---|---|---|---|---|
| Activity | high | middle | low | high | high | high |
| Pd loading [%] | 5 | 5 | 5 | 4.63 | 4.63 | 4.63 |
| BET surface area [m2/g] | 764 | 758 | 789 | 604 | 654 | 717 |
| Pore volume [cm3/g] | 0.60 | 0.61 | 0.63 | 0.47 | 0.50 | 0.55 |
| Grain size Dn10 [μm] | 0.754 | 0.728 | 0.943 | — | — | — |
| Dn50 [μm] | 1.05 | 1.09 | 1.44 | — | — | — |
| Dn90 [μm] | 2.66 | 3.07 | 4.18 | — | — | — |
| Pd dispersion [%] | 24 | 23 | 30 | 21 | 24 | 24 |
| Pd surface area [m2/g] | 5.3 | 5.1 | 6.6 | 4.2 | 4.9 | 5.0 |
| Pd particle size (hemi-sphere, | 4.7 | 4.8 | 3.8 | 5.5 | 4.7 | 4.6 |
| chemisorption) [nm] | | | | | | |

The BET surface area for S1, S2 and S3 is similar, since the same carbon support was used. In case of the films, comprising PTFE in fibrillated form, the porosity and the surface area were dependent on the absolute film thickness, showing a slight decrease in surface area for lower thickness. The effect was limited to maximum of 20% surface area loss for the thinnest catalyst films of 100 μm.

The Pd dispersion is similar for the S1 and S2 and higher for S3 (see Table 2). In case of F1, F4 and F7, the Pd dispersion was retained independently from the film thickness (considering the margin of the experimental error of the chemisorption measurements).

The typical structure of the films, comprising PTFE in fibrillated form, was elucidated by SEM (FIG. 3, 4), where PTFE nanofibrils were observed holding together the active carbon grains (solid catalyst particles). The Pd-dispersion was investigated by acquiring SEM images in backscattering mode and by analyzing the catalyst powder by TEM. TEM images did not show remarkable difference in Pd-distribution among the three catalysts.

### II. Application and Kinetic Analysis of Films, Comprising PTFE in Fibrillated Form and Pd on C as Solid Catalyst Particles, in Batch Autoclave

Hydrogenation of nitrobenzene (NB) to aniline was chosen as a test reaction for the catalytic systems.

### Method:

A 60 ml batch reactor was used to evaluate the mass transfer/diffusion phenomena in the original catalyst powders and in the films, comprising PTFE in fibrillated form and solid catalyst particles. The autoclave contained a magnetically coupled stirrer and stream breakers to provide good mixing and minimize gas/liquid mass transport limitations. For kinetic studies the following procedure was developed. The catalyst powder (Pd/C), solvent (methanol) and hydrogen were fed in the autoclave (5 barg), while a solution of nitrobenzene was inserted in the charger. The autoclave was stabilized at the desired temperature. The reaction started at to when the valve of the charger was opened, and nitrobenzene inserted into the reactor (final nitrobenzene concentration 0.03 mol L⁻¹). The hydrogen consumption of the reaction is calculated by recording the variation of the pressure in the autoclave versus time. Knowing this, the hydrogen moles consumed and the variation in nitrobenzene concentration are calculated. The reaction rate is reported as mol s⁻¹ of the converted nitrobenzene normalized for the Pd mass. The Arrhenius plot and the effectiveness factor were used to compare the powder and the films, comprising PTFE in fibrillated form and solid catalyst particles, for a temperature range from −8° C. to 20° C. (range where data for the catalyst powders were obtained in the kinetic regime). In this study, the effectiveness factor (η) is defined as the ratio between the reaction rate observed in the films and the reaction rate of the powder.

The reaction rate decreased for high film thickness and consequently with it the effectiveness factor for the respective films (Table 3). For definition, the effectiveness factor of the catalyst powder in kinetic regime is 100%. In Table 3, the highest active catalyst powder S1 shows an effectiveness factor less than 8%, which means that less than 8% of the porous body is effectively used, showing therefore mass transfer limitations. For the middle (F2, F5, F8) and low active (F3, F6, F9) catalysts the effectivity factor is higher (40-47% for the 100 μm film, Table 3), but still far from 100%.

Sandwich films were considered as approaches to increase the effectivity of monolayer films. The 100 μm sandwich film (low activity catalyst) had an enhanced activity, comparable to the powder, demonstrating therefore that using this geometry the noble metal can be fully exploited and no mass transfer limitation were present.

TABLE 3
Effectiveness factor (η) calculated for the reaction
nitrobenzene to aniline over films comprising solid catalyst
particles (S1, S2, S3) and PTFE in fibrillated form.

| Film | η at −8° C. | η at 0° C. | η at 10° C. | η at 20° C. |
|---|---|---|---|---|
| F1 | 7.3% | 6.2% | 5.0% | 4.2% |
| F4 | 3.1% | 2.6% | 2.2% | 1.9% |
| F7 | 3.2% | 2.8% | 2.4% | 2.1% |
| F2 | 47.3% | 36.5% | 26.9% | 20.2% |
| F5 | 16.6% | 16.7% | 16.7% | 16.8% |
| F8 | 13.3% | 13.3% | 13.3% | 13.3% |
| F3 | 40.9% | 35.8% | 30.7% | 26.5% |
| F6 | 13.7% | 13.4% | 13.0% | 12.6% |
| F9 | 10.6% | 10.2% | 9.9% | 9.5% |
| F10 | 100% | 100% | 90.8% | 76.3% |

### III. Films, Comprising PTFE in Fibrillated Form and Pd on C as Solid Catalyst Particles, Application and Kinetic Analysis of Films, in Microreactor as Flow Chemistry Application

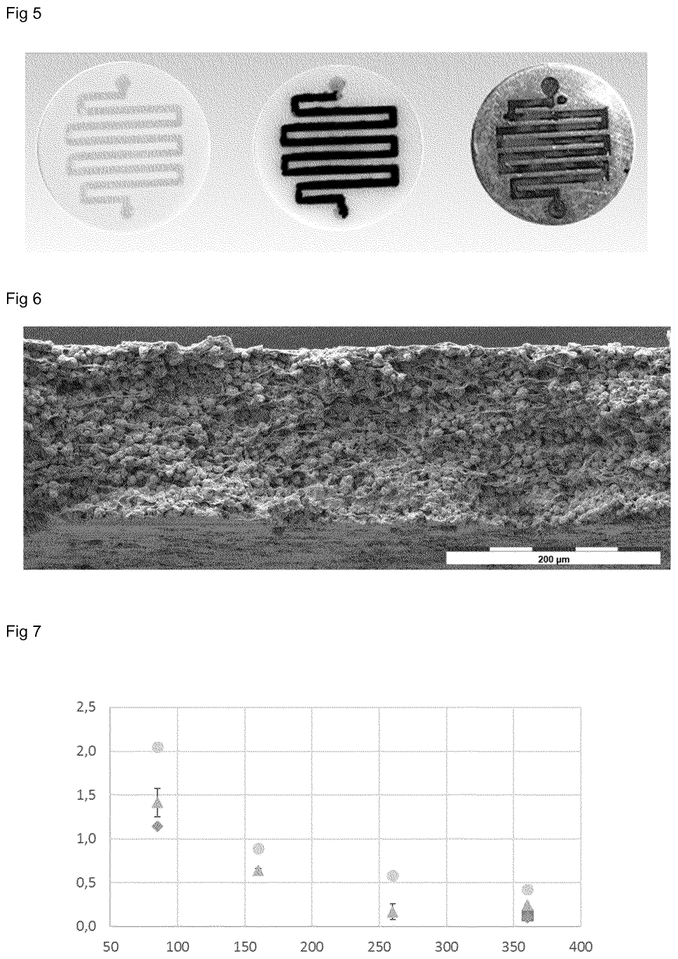

Films, comprising PTFE in fibrillated form and Pd on C as solid catalyst particles as described above, were immobilized on the microchannels of a 100 μl microreactor and tested in continuous mode. The film F1 was inserted in the microstructures by pressing it gently and going to constitute one of the wall of the channel (FIG. 5). Hydrogenation of nitrobenzene was used as test reaction to monitor the catalyst performance over time.

FIG. 5: Microchannels used in the conversion of nitrobenzene to aniline. On the left, PTFE microchannels, on the center PTFE microchannel with immobilized films, comprising PTFE in fibrillated form and Pd on C as solid catalyst particles, on the right stainless steel microchannels with said film and with Taylor flow applied.

Method: The nitrobenzene solution (0.03 mol/l in methanol, liquid flow 2 ml/min) was supplied by a syringe pump. This was mixed with hydrogen (approximative volume ratio liquid/gas 1:5) by a T junction and insert consequently in the microreactor. The reaction was carried out at 20° C. at atmospheric pressure with an approximate residence time of circa 5 s.

In FIG. 5, the microchannels are shown: on the left empty microchannel, on the center with the immobilized film, comprising PTFE in fibrillated form and Pd on C as solid catalyst particles, and on the right Taylor flow was applied on said film. The residence time was chosen to hit a conversion a value lower than 100% in order to monitor eventual deactivation. The conversion was approximatively stable at around 50% during a TOS (time on stream) of 5 hours (see Table 4).

The selectivity toward aniline was also high (near 100%), as observed by UV-Vis spectrometry and confirmed also by GC-MS. The linear combination of UV-Vis spectra of nitrobenzene and aniline could fit properly the product spectrum, being therefore able to determine the aniline concentration. In conclusion, films, comprising PTFE in fibrillated form and Pd on C as solid catalyst particles, were suitable also for a) continuous operation and b) operation in microreactors.

TABLE 4
Conversion of nitrobenzene to aniline in a microreactor
(reaction conditions: 20° C., 1 atm H₂, 0.03M
nitrobenzene, residence time 5 s).

| Time on stream [h] | Conversion [%] |
|---|---|
| 0.5 | 57.2% |
| 1.0 | 56.0% |
| 1.5 | 57.9% |
| 2.0 | 47.4% |
| 2.5 | 50.1% |
| 3.0 | 44.1% |
| 3.5 | 51.9% |
| 4.0 | 53.7% |
| 4.5 | 53.9% |
| 5.0 | 49.6% |
| 5.5 | 51.3% |

### IV. Additional Investigative Experiments

Additional experiments were carried out in the autoclave to assess:
Resistance of the film to high temperatures (180° C.) and leaching of Pd
Influence of the binder amount on catalytic performance
Reusability of the films comprising solid catalyst particles in sequential batch reactions

After the film (low activity catalyst, 100 μm) was exposed at 180° C. for 3 h in methanol, the autoclave was cooled down and a reaction was performed at 20° C. Same procedure was applied for a powder catalyst sample, treated in the identical way.

Both resulted catalytically not active materials (powder catalyst and catalyst film) after the thermal treatment at 180° C. We assume that the powder catalyst was deactivated during the treatment procedure. However, the film looked macroscopically intact by visual inspection and SEM, indicating that the fibrils were intact and mechanically stable. Therefore, the deactivation processes seem to have only affected the catalytic function and not the mechanical stability. In conclusion, deactivation processes are only dependent on the nature of the catalyst and not on the fibrillation process. As well, leaching at different temperatures up to 180° C. was investigated. No leaching could be observed. The content of Palladium in the solution was under the detection limit of the instrument.

Films with a different amount of fibrillated PTFE (20% instead 7.5% of PTFE) were tested and no significant differences could be seen in the catalytic activity. This result constitutes an advantage in term of flexibility in tuning the catalyst surface property, varying the polarity of the film (larger amounts of PTFE as binder will result in a less polar catalyst film).

A sequence of repeated reactions was performed using the same film F3 (low activity catalyst, 100 μm). The activity for a set of four experiments decrease compared to the first experiment to 90% for the second, 75% for the third and 59% for the fourth experiment. Preliminary experiments with the powder suggested that also the powder is affected by deactivation. In conclusion, deactivation processes are attributed to the nature of the catalytical powder and not to the specific immobilization in the films, comprising PTFE in fibrillated form and Pd on C as solid catalyst particles.

### V. Mechanical Stability Experiments

Additional experiments were carried out investigating the mechanical stability of catalytic fibrillated films at the example of transition metal carbonate particles with a d50 of 9 μm and a BET surface 3.3 m²/g (90%) and PTFE particles with a d50 of 400 μm (10%).

To investigate the effect of multiple layers to the film stability, the film samples were folded like described in the following:

The initial films all were single layered with a thickness of 360 μm. Those films were produced as described above. First the catalyst particles (transition metal carbonate) were mixed with PTFE particles. Second the particle mixture was pre-fibrillated for 10 min via ball mill (1-liter container on rolling bench, 15 g particles, 1 kg of 2.7 mm zirconium oxide milling balls). The resulting particle/PTFE flakes were finally calendered between two rollers and a fixed gap of 360 μm. 2-layer films were achieved by folding the single-layer films before compacting them again to 360 μm via calendering rolls. Accordingly, 4-layer films were achieved by folding 360 μm thick 2-layer films before compacting them again to 360 μm thickness. 8-layer films were achieved similar, starting with 360 μm thick 4-layer films.

For investigating the effect of further compaction to the film stability, the multiple layered films were further compacted, starting from their common thickness of 360 μm.

For measuring the mechanical stability of the films, a minimum of three samples, each 10×50 mm², were measured for tensile strength applying a force testing machine BTC-FR2.5TN.D09 from Zwick GmbH.
FIG. 6: SEM-cross-section view of a 160 μm thick, 4-layered film comprising transition metal carbonate particles (90%) and PTFE (10%).
FIG. 7: Plot of tensile strength measurements for various layered and compacted films comprising transition metal carbonate particles (90%) and PTFE (10%). The square (▪) represents the initial 1-layered film at 360 μm without any compaction or folding. The diamonds (▴) represent the tensile strength of compacted 2-layer films, the triangles (♦) represent the tensile strength of compacted 4-layer films, while the circles (●) represent the tensile strength of compacted 8-layer films.

In FIG. 6 a SEM-cross-section view of a 160 μm thick, 4-layered film is shown. In the picture there is no layered structure visible. Hence, the layered processing of the films vanishes during further compaction which promotes further fibrillation between the layers.

In FIG. 7 the results of the tensile strength measurements are plotted. Two overall trends are obvious. First, the tensile strength for all amounts of layers increases when the films get compacted from 360 μm to 85 μm. Second, the tensile strength increases with an even higher impact when the number of layers is increased from 2 to 8. This is due to fact that the layering is achieved by a folding and a subsequent compaction to the initial thickness.

1.-9. (canceled)

11. A process for carrying out a chemical reaction in a chemical reactor, in which at least one starting material, which is an organic chemical compound comprising 1 to 80 carbon atoms, is converted into at least one reaction product in a fluid phase in the presence of a film comprising solid catalyst particles, which catalyze said chemical reaction, and comprising an organic polymer in fibrillated form, wherein the film comprises at least one layer comprising solid catalyst particles and the organic polymer in fibrillated form, wherein the mass fraction of the organic polymer in fibrillated form in said layer is in the range from 0.06 to 0.2 and the mass fraction of solid catalyst particles in said layer is in the range from 0.8 to 0.94 based on the total weight of said layer, wherein the organic polymer is a fluoropolymer, and wherein the mass fraction of the sum of the starting material and of the reaction product based on the total mass of the fluid phase is in the range from 0.01 to 1.

12. The process according to claim 11, wherein the chemical reaction is selected from the group of chemical reactions consisting of oxidations, reductions, substitutions, additions, eliminations and rearrangements.

13. The process according to claim 11, wherein the chemical reaction takes place at a temperature in the range from −78° C. to 350° C.

14. The process according to claim 11, wherein the chemical reactor is a fixed-bed reactor selected from the group of reactors consisting of tubular reactors, adiabatic reactors, multitube reactors and microreactors.

15. The process according to claim 11, wherein the fluid phase is a liquid phase.

16. The process according to claim 11, wherein the solid catalyst particles have a particle size d50 in the range from 0.1 to 1000 μm.

17. The process according to claim 11, wherein the film has a thickness in the range from 0.1 μm to 20000 μm.

18. The process according to claim 11 wherein the thickness of the layer comprising solid catalyst particles and the organic polymer in fibrillated form is in the range from 1 μm to 200 μm.

19. The process according to claim 11, wherein the film comprises at least two layers of different compositions, wherein at least one of the two outer layers of the film is the layer comprising solid catalyst particles and the organic polymer in fibrillated form.

20. The process according to claim 11, wherein at least one part of the film provides a porosity of 5 to 70%.

21. A process for carrying out a chemical reaction in a chemical reactor comprising:
converting at least one starting material, which is an organic chemical compound comprising 1 to 80 carbon atoms, into at least one reaction product in a fluid phase in the presence of a film comprising solid catalyst particles, which catalyze said chemical reaction, and comprising an organic polymer in fibrillated form, wherein the film comprises at least one layer comprising solid catalyst particles and the organic polymer in fibrillated form, wherein the mass fraction of the organic polymer in fibrillated form in said layer is in the range from 0.06 to 0.2 and the mass fraction of solid catalyst particles in said layer is in the range from 0.8 to 0.94 based on the total weight of said layer, wherein the organic polymer is a fluoropolymer, and wherein the mass fraction of the sum of the starting material and of the reaction product based on the total mass of the fluid phase is in the range from 0.01 to 1.
